(19) United States
(12) Patent Application Publication
(10) Pub. No.: US 2015/0174392 A1
(43) Pub. Date: Jun. 25, 2015

(54) Gel Pad Dispenser

(71) Applicant: Sunstar Suisse Sa, Etoy (CH)

(72) Inventors: Tomas Stringdahl, Halmstad (SE); Christian Christiansen, Birkerod (DK)

(21) Appl. No.: 14/410,678

(22) PCT Filed: Jun. 28, 2013
(86) PCT No.: PCT/EP13/63663

§ 371 Date: Dec. 23, 2014

Related U.S. Application Data
(60) Provisional application No. 61/666,035, filed on Jun. 29, 2012.

(51) Int. Cl. A61N 1/04

(52) U.S. Cl. CPC A61N 1/0492; A61N 1/0452

(57) ABSTRACT

The present invention relates to a dispenser for arranging an adhesive element on a medical device, in particular a dispenser for arranging an adhesive gel pad on an electrode assembly. One embodiment discloses a dispenser for arranging an adhesive element on a medical device, said dispenser comprising a holder adapted to receive the medical device, a delivery mechanism comprising a stack of said adhesive elements and a resilient element, the stack arranged on the resilient element such that the stack of adhesive elements is pressed towards the holder, each of said adhesive elements arranged between layers of removable protective sheet, and a traction mechanism comprising a traction element connected to the removable protective sheet and a switch adapted for operating the traction element, said traction mechanism adapted for pulling said removable protective sheet upon activation of the switch.

The present invention relates to a dispenser for arranging an adhesive element on a medical device, in particular a dispenser for arranging an adhesive gel pad on an electrode assembly. The present disclosure further relates to a disposable gel pad tray for this dispenser. The present disclosure further relates to a medical device and an inter-related adhesive element and a kit of said parts.

### BACKGROUND OF INVENTION

Medical devices for monitoring or applying electric energy to the body are generally known in the art. E.g. muscles can be monitored, e.g. for diagnostic purposes, by measuring the electric signals involved in muscle contraction or they can be stimulated, e.g. for therapeutic purposes, by applying electrical signals to the skin. This monitoring and stimulation can be provided by means of electrodes and in order to ensure contact with the skin the electrodes can be provided with an adhesive and conductive material. Electrically conductive adhesive solid hydrogels and liquid gels can provide this electrical interface to the skin. The conductive material can be applied to the electrode at the electrode manufacturer such that the material cannot be removed from the electrode and reused. However, once the electrode with conductive material has been in contact with a patient, it is generally not desirable to apply the same electrode with the same conductive material to a different patient. Thus, for the electrodes to be reusable the electrodes and the conductive material must be configured such that the conductive material can be applied and removed and new conductive material re-applied when needed. This operation is typically purely manual and with medical devices for home use, it may even be the patient that applies the conductive material. To assist the user in applying the conductive material it is often formed as adhesive elements, such as gel pads, with a circumferential shape that matches the shape of the electrodes such that they can be easily applied. In spite of this, it may be undesirable for the patients to perform this application of gel pads and furthermore, it may be of utmost importance that the gel pads are placed at exactly the correct position for the medical device to function optimal.

### SUMMARY OF INVENTION

A dispenser for automatically applying gel pads to electrodes is disclosed in WO 2009/015074 where the gel pads are provided in a roll with the gel pads interposed between two protective liners to form a tape. Two take-up rollers work to peel off the liners from the outermost gel pad in the roll just before meeting the electrode. The electrode with the gel pad subsequently passes between two pressure rollers thereby facilitating an adhesive bond between gel pad and electrode. Thus, this prior art dispenser “rolls” the gel pad onto the electrode. However, many medical devices are indeed not suited for being squeezed between two pressure rollers, e.g. if the electrodes form an integral part of the medical device. And it may be a problem that with the gel pads located in a roll, movement of the gel pad is necessarily involved when applying a gel pad to an electrode, thereby increasing the risk of incorrect placement on the electrode.

One embodiment of the invention therefore relates to a dispenser for arranging an adhesive element on a medical device, said dispenser comprising a holder adapted to receive the medical device, a delivery mechanism comprising a stack of said adhesive elements and a resilient element, the stack arranged on the resilient element such that the stack of adhesive elements are pressed towards the holder, each of said adhesive elements arranged between layers of removable protective sheet, and a traction mechanism comprising a traction element connected to the removable protective sheet and a switch adapted for operating the traction element, said traction mechanism adapted for pulling said removable protective sheet upon activation of the switch. This dispensing device ensures the correct use of the medical device because fresh adhesive elements can be applied at a predetermined location without the user's interference, because the dispenser is preferably adapted to automatically or at least semi-automatically arrange the adhesive element at a predetermined position at the medical device.

A further embodiment of the invention relates to a tray for the abovementioned dispenser comprising a stack of said adhesive elements arranged on a resilient element, said adhesive elements arranged in between layers of protective sheet, and a traction element connected to the protective sheet and adapted to pull said protective sheet. The tray is preferably replaceable and/or disposable.

A further embodiment of the invention relates to a method for arranging a disposable adhesive element on a medical device comprising the steps of:
activating the switch of the dispenser as herein disclosed thereby releasing the protective sheet from one side of the topmost adhesive element in the stack,
arranging the medical device in the holder of said dispenser, the topmost adhesive element thereby being attached to the medical device, and
removing the medical device with the adhesive element from the dispenser.

The first two steps may be carried out in reverse order, i.e. the medical device is arranged in the holder before the switch is activated. Thus, a further embodiment of the invention relates to a method for arranging a disposable adhesive element on a medical device comprising the steps of:
arranging the medical device in the holder of the dispenser as herein disclosed,
activating the switch thereby releasing the protective sheet from the side of the topmost adhesive element in the stack that faces the medical device, the topmost adhesive element thereby being attached to the medical device, and
removing the medical device with the adhesive element from the dispenser.

A further aspect of the invention relates to a medical device for monitoring muscle activity of an individual comprising an electrode assembly with a plurality of electrodes, preferably three electrodes, the electrode assembly comprising a part of an attachment mechanism adapted to match a corresponding part of an attachment mechanism on an adhesive element, said adhesive element adapted for being applied to the skin of said individual and provide electrical contact between the skin and the electrode assembly, wherein the medical device is adapted for:
receiving and monitoring electrical signals, and
providing electrical stimulation to said individual.

A further embodiment relates to the interrelated adhesive element comprising a part of an attachment mechanism adapted to match a corresponding part of an attachment mechanism on a medical device for monitoring muscle activity of an individual, wherein the adhesive element is adapted for being applied to the skin of said individual and provide electrical contact between the skin and the medical device.

A further embodiment relates to a kit comprising the abovementioned medical device and adhesive element which are adapted to be attached to each other by means of the attachment mechanism.

### DESCRIPTION OF DRAWINGS

The invention will now be described in further details with reference to the drawings in which

FIG. 1a shows an illustration of one embodiment of the dispenser according to the invention,

FIG. 1b is a cut-through illustration of the dispenser,

FIG. 1c shows an example of a stack of adhesive elements located on a spring,

FIG. 1d shows an example of a stack of adhesive elements connected to a traction element,

FIG. 2 shows an example of a switch engaging a traction element,

FIG. 3a shows various illustrations of the present dispenser,

FIG. 3b is an illustration of the dispenser, and

FIGS. 4a-b show illustrations of an adhesive element and a medical device with snap locks to ensure the attachment.

### DETAILED DESCRIPTION OF THE INVENTION

The medical device may be an electrode assembly adapted to be arranged on the skin of a user. The medical device may be a device for monitoring muscle activity of an individual comprising an electrode assembly with a plurality of electrodes, wherein the medical device is adapted for receiving and monitoring electrical signals, and providing electrical stimulation to said individual. Examples of such medical devices can be seen in PCT/DK2004/00223 (published as WO 2004/87258), PCT/DK2008/050230 (published as WO 2009/36769), and PCT/Dk2008/050231 (published as WO 2009/36770).

In a further embodiment the adhesive element is arranged on an electrode assembly of the medical device, i.e. the electrode assembly may be an external or integral part of the medical device. The shape of the adhesive element may be formed to match the circumferential shape of the medical device or match the circumferential shape of an electrode assembly of the medical device. The adhesive element is preferably adapted to form an adhesive and/or conductive bond between the medical device and the skin of a user.

An advantage of the dispenser according to the present invention is that the medical device and the stack of adhesive elements are fixed in relation to each other. This helps to ensure the correct use of the medical device because fresh adhesive elements can be applied at the desired location on the medical device. When operating the dispenser the user only removes the protective sheet from an adhesive element without interfering with the positions of the medical device and the adhesive elements. Further, the present dispenser may be fully manually driven without depending on an electrical input.

A further advantage is that the adhesive elements are arranged in stack. Compared to a roll of adhesive elements, a stack of elements ensures that the adhesive elements are not preformed into an arc due to the curvature of the roll.

The adhesive elements may be replaceable or disposable. The adhesive elements may be pads, such as gel pads. The adhesive element may be adapted to form a conductive connection between the medical device and the skin of a user. Thus, the adhesive elements may comprise conductive material, such as a conductive gel. A conductive gel is a polymeric material that when applied to the surface of the electrode permits the flow of electric current from the electrode through the material to the subject when in contact with the subject. The conductive gel may be part of a gel pad.

In one embodiment of the invention the traction mechanism is adapted for releasing the protective sheet from at least one side of an adhesive element upon activation of the switch, preferably the topmost or outermost adhesive element in the stack. I.e. the traction element is adapted for peeling off the protective sheet thereby exposing one side of the adhesive element.

In one embodiment of the invention the traction element comprises a cylindrical reel for wounding the protective sheet. A rotation of the cylindrical reel thereby pulls the protective sheet.

In one embodiment of the invention the switch is adapted to engage the traction element, e.g. mechanically engaging the traction element. This may be provided by one or more racks adapted to engage the traction element. Correspondingly the traction element may comprise one or more toothed wheels, e.g. adapted to engage the racks. The toothed wheel may be provided at the ends of a cylindrical reel. I.e. engaging the toothed wheels with the racks rotates the cylindrical reel and pulls the protective sheet. Furthermore, said one or more toothed wheels may be adapted to freewheel in one direction of rotation.

In a further embodiment of the invention the switch comprises a slidable button and wherein activation of the switch includes sliding the slidable button. Activation of the switch may correspond to a single slide with the slidable button, two or more slides with the slidable button or sliding back and forth with the slidable button. Furthermore, the switch may be adapted to engage the traction element for only one sliding direction of the sliding button.

In a further embodiment of the invention the traction mechanism further comprises a resilient switch release, e.g. the resilient switch release is adapted to return the switch to a starting point after activation of the switch.

The traction mechanism may be manually operated and driven, i.e. the actuating force and the energy required to pull the protective sheet is provided by the user's movements. However, the traction mechanism may also be at least partly electronically driven. E.g. the traction mechanism may comprise a motor or actuator for driving the traction element.

In one embodiment of the invention the stack of adhesive elements is located adjacent the holder. E.g. the stack of adhesive element may be located vertically below said holder or the stack of adhesive element may be located horizontally adjacent said holder. Furthermore, the resilient element may comprise one or more springs. Thus, the stack of adhesive elements can be located on one or more (compressed) springs to force the stack towards the holder.

The dispenser and/or the tray may be provided with a rubber packing, e.g. in the form of a rubber ring, around the opening between holder and adhesive elements. This is to ensure an airtight sealing of the stack of adhesive elements such the adhesive material of the adhesive elements does not dry out.

### Medical Device

As stated above a further aspect of the invention relates to a medical device for monitoring muscle activity of an individual comprising an electrode assembly with a plurality of electrodes, preferably three electrodes, the electrode assembly comprising a part of an attachment mechanism adapted to match a corresponding part of an attachment mechanism on an adhesive element (e.g. the herein mentioned adhesive element), wherein the medical device is adapted for receiving and monitoring electrical signals and providing electrical stimulation to said individual. Examples of such medical devices can be seen in PCT/DK2004/00223 (published as WO 2004/87258), PCT/DK2008/050230 (published as WO 2009/36769), and PCT/Dk2008/050231 (published as WO 2009/36770), which are hereby incorporated by reference in their entirety.

The medical device may be suitable for monitoring muscle activity related to bruxism, and may thus be adapted for processing the received signals in order to detect said bruxism, and the electrical stimulation may therefore be a feedback signal provided in response to detection of the bruxism. The electrodes may be arranged in a fixed spatial relationship one to another. The electrodes may be mounted on a common substrate.

### Adhesive Element

A further embodiment of the invention relates to an adhesive element comprising a part of an attachment mechanism adapted to match a corresponding part of an attachment mechanism on a medical device for monitoring muscle activity of an individual (e.g. the abovementioned medical device), wherein the adhesive element is adapted for being applied to the skin of said individual and provide electrical contact between the skin and the medical device.

The adhesive element(s) may be double adhesive, i.e. provided with adhesive material on both sides. I.e. adhesive material provided the connection between the adhesive element the medical device and between the adhesive element and the skin of the user. However, the adhesive element may be attached to the medical device by other means. Thus, in a further embodiment of the invention the adhesive element(s) is adapted to match an attachment mechanism on the medical device. E.g. if the attachment mechanism on the medical device comprises one or more clips or clamps adapted to grab and thereby hold the adhesive element.

In a further embodiment of the invention the adhesive elements are adhesive on one side and comprise a part of an attachment mechanism on the opposite side, said part of said attachment mechanism adapted to match a corresponding part of an attachment mechanism on the medical device. In a further embodiment of the invention said part of said attachment mechanism on the adhesive elements comprises one or more components that are shaped to match a corresponding component of the attachment mechanism on the medical device. E.g. the attachment mechanism may e.g. be one, two, three or more snap locks. Furthermore, the shape of said components may be polygonal, such as triangular, rectangular, pentagonal, hexagonal, or star-shaped. Matching components may be a protruding part shaped to match a corresponding groove.

The adhesive element is preferably at least partly conductive so as to provide the electrical contact between the skin and the medical device via the electrode assembly. Thus, the adhesive element may be having contact areas for electrical connection with the skin which is spaced from one another, e.g. corresponding to the spacing between the electrodes in the electrode assembly. The contact areas are preferably spaced from one another by at least 2 mm.

In a further embodiment the attachment mechanism comprises hook and loop fasteners. Furthermore, the attachment mechanism may comprise one or more clips or clamps. Even further, the attachment mechanism may be at least partly magnetic to ensure attraction between medical device and adhesive element. One advantage of using an attachment mechanism is that it may help to ensure the correct attachment between medical device and adhesive element, because the geometrical configuration of the attachment mechanism will typically ensure a correct attachment. Furthermore, the attachment mechanism may be adapted to provide an electrical connection between the adhesive element and the medical device, e.g. by providing a metallic attachment mechanism.

The attachment mechanism may be adapted to allow a certain migration between the corresponding parts, preferably a migration that maintains any electrical connection provided by the attachment mechanism. The migration may be provided in a direction substantially perpendicular to the skin when the medical device with adhesive element is applied to an individual. By allowing a certain migration in this direction the adhesive element may be allowed to at least partly adapt to curvatures of the individual, e.g. when the medical device is applied to monitor the temporalis muscle. The medical device itself is typically not flexible or bendable but by allowing the part that has the contact with the skin (i.e. the adhesive element, which is typically flexible) to at least partly form to the skin of the individual a better contact of the skin of the individual may be provided and thereby an improved monitoring of muscle activity. Migration may for example be provided in a snap lock, which may be adapted such that the protruding part of the snap lock is allowed to migrate in the corresponding groove without breaking the attachment and contact between the parts. An attachment mechanism migration may be a few millimetres, such as between 0.1 and 10 mm, preferably at least 1, 2, 3, 4, 5 mm or at least 6 mm, or less than 10, 9, 8, 7, 6, 5, 4, 3, 2 mm or less than 1 mm.

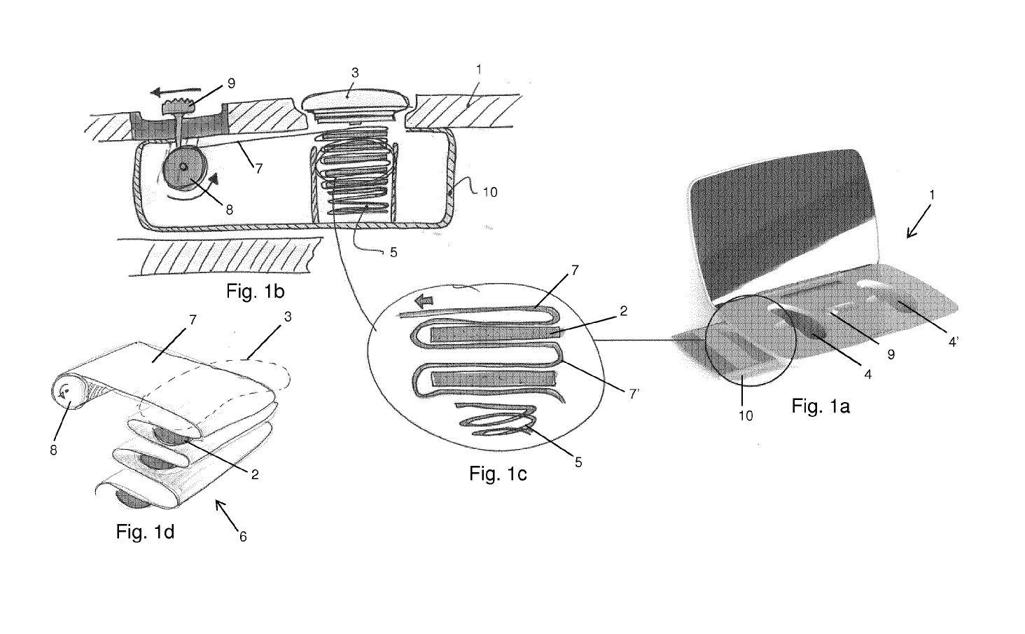

A stack of adhesive elements may be formed of adhesive elements sandwiched between layers of the protective sheet. The protective sheet may form a band, i.e. a protective liner. The stack of adhesive elements may be formed such that the band of protective sheet can be released from the topmost or outermost adhesive element by pulling the band from only one side of the stack. This may be provided by arranging each of said adhesive elements between two layers of protective sheet, e.g. the two layers may be formed by folding a single layer of protective sheet, see FIG. 1 for an illustration of the principle.

The adhesive elements may be arranged exactly on top of each other in said stack. Thereby the width and depth of the stack will be substantially the same as the adhesive elements. However, in a further embodiment the adhesive elements are displaced in relation to each other in said stack. With e.g. an attachment mechanism on the adhesive elements the height of said attachment mechanism may require that the adhesive elements are displaced, preferably displaced transversally, in relation to each other in the stack.

A further embodiment relates to a kit comprising the abovementioned medical device and adhesive element which are adapted to be attached to each other by means of the attachment mechanism.

### EXAMPLES

The GrindCare from Medotech NS is an example of a medical device provided with electrodes that needs to be supplied with fresh gel pads for every use. The user typically wears the GrindCare during the night. The electrodes of the GrindCare must be placed on the skin of the user to be in contact with the muscles of the temple. The dispenser according to the present invention helps the user to automatically place gel pads correctly on the GrindCare electrodes before use, i.e. typically in the evening before going to bed.

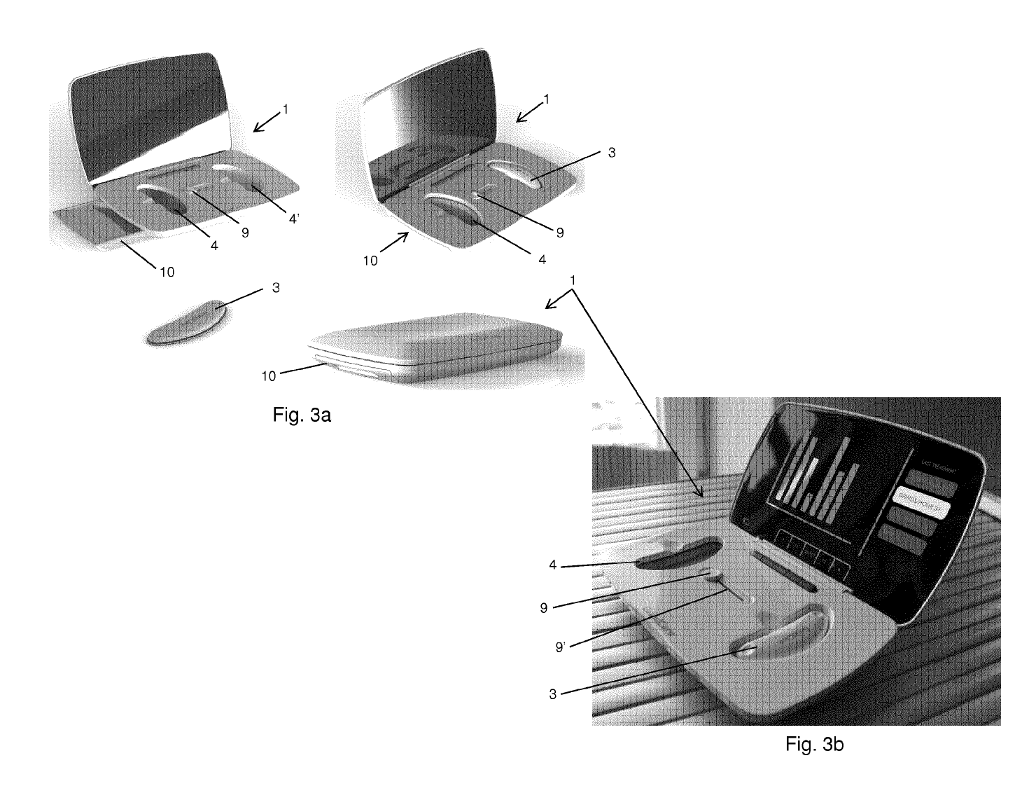

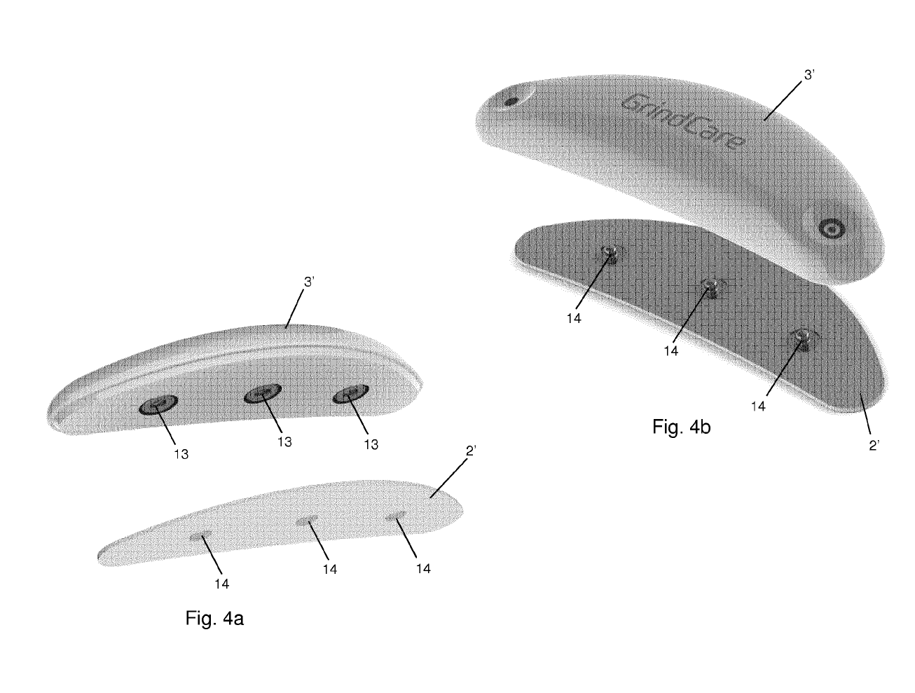

An exemplary dispenser 1 according to the present invention is illustrated in FIGS. 1a, 3a and 3b. The medical device 3 is shown in FIGS. 3 and 4. Electrodes are provided as an integral part of the device 3. They are located at the bottom of the device 3 and can be seen in FIG. 4a. When fresh gel pads 2 are to be arranged on the device 3 it is placed in the holder 4 in the dispenser 1. In the drawings the dispenser is part of a “suitcase” for the device 3 with a display and control buttons. An additional holder 4′ is provided for charging the battery driven device 3. As seen in FIG. 1a a tray 10 is open in the left side of the device 1. The tray 10 houses a stack 6 of adhesive elements 2 (gel pads) sandwiched between layers of protective sheet 7. The protective sheet 7 forms a band and two adjacent gel pads are separated by two layers of protective sheet, because the sheet 7 is folded 7′ between two gel pads. Consequently the protective sheet 7 can be released from the topmost gel pad in the stack 6 by pulling from only one side. The protective sheet 7 is connected to a traction element in the form a cylindrical reel 8. Thus, by rotating the reel 8 the sheet 7 can be pulled. A resilient element in the form of a spring 5 located below the stack 6. The (compressed) spring 5 forces the stack 6 towards the device 3 as seen in FIG. 1b. When the reel 8 is rotated the sheet 7 is released from the topmost gel pad 2 and the compressive force of the spring will force the adhesive gel pad 2 towards the device 3 to form an adhesive bond between gel pad 2 and device 3.

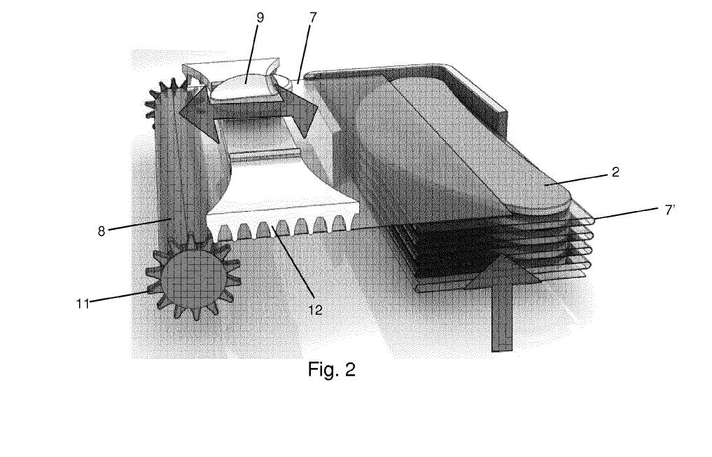

The traction element 8 is activated by means of a switch, in this case in the form of a slidable button 9 as most clearly seen in FIG. 3b where the sliding passage 9′ for the switch 9 is visible. The engagement between switch 9 and traction element is illustrated in FIG. 2 where the cylindrical wheel 8 is provided with toothed wheels 11 at the two ends. The switch 9 is provided with two racks 12 adapted to engage the toothed wheels 11. When the switch 9 is slit in the passage 9′ the racks 12 engage the toothed wheels 11 to rotate the reel and release the protective sheet 7 from the gel pad 2. With this slidable switch 9 it is a movement induced by the user that releases the sheet 7 and bonds the gel pad 2 to the device 3, i.e. without requiring electrical energy. The dispenser 1 is adapted such that a single slide back and forth with the switch 9 releases the protective sheet 7 from the topmost gel pad 2. The traction element may be adapted such that the toothed wheels 11 can freewheel when the switch 9 is slit back to starting position, i.e. freewheeling without rotating the reel 8. Or the switch 9 may be adapted such that the racks 12 disengage the toothed wheels 11 when returning to the starting position, e.g. by slightly adjusting the position of the racks 12 relative to the toothed wheels 11.

This tray 10 may be replaceable or disposable or refillable. Thus, when an entire stack of gel pads has been used the tray 10 is retracted and replaced with a new tray where a fresh stack of gel pads is arranged.

The adhesive elements in the stacks in FIGS. 1 and 2 are double adhesive. However, as illustrated in FIGS. 4a and 4b the attachment between medical device 3′ and adhesive element 2′ may be provided by means of an attachment mechanism, in this case three metallic snap locks 13, 14. The protruding part 14 of the snap locks is located on the adhesive element 2′ in FIG. 4, however it may just as well be located on the medical device. The snap lock part 13 on the medical device 3′ also forms the electrode of the medical device. As also seen from FIG. 4 the geometrical configuration of the snap locks 13, 14 ensure the correct placement of the adhesive element 2′ relative to the medical device 3′. It can further be seen from the FIG. 4 that a snap lock may be configured to allow a certain migration of the protruding part 14 inside the groove part 13, such that the adhesive element 2′ can be slightly bended (convex or concave) to at least partly follow the curvature of the skin of an individual without losing the connection between the parts 13, 14.

### Further Details of the Invention

The invention will now be described in further detail with reference to the following items:
1. A dispenser for arranging an adhesive element on a medical device, said dispenser comprising
   a holder adapted to receive the medical device
   a delivery mechanism comprising a stack of said adhesive elements and a resilient element, the stack arranged on the resilient element such that the stack of adhesive elements is pressed towards the holder, each of said adhesive elements arranged between layers of removable protective sheet, and
   a traction mechanism comprising a traction element connected to the removable protective sheet and a switch adapted for operating the traction element, said traction mechanism adapted for pulling said removable protective sheet upon activation of the switch.
2. The dispenser according to item 1, wherein the traction mechanism is adapted for releasing the protective sheet from at least one side of an adhesive element, preferably upon activation of the switch.
3. The dispenser according to any of the preceding items, wherein the traction mechanism is adapted for releasing the protective sheet from at least one side of the topmost adhesive element in the stack, preferably upon activation of the switch.
4. The dispenser according to any of the preceding items, wherein the switch is adapted to engage the traction element.
5. The dispenser according to any of the preceding items, wherein the switch mechanically engages the traction element.
6. The dispenser according to any of the preceding items, wherein the switch comprises one or more racks adapted to engage the traction element.
7. The dispenser according to any of the preceding items, wherein the traction element comprises a cylindrical reel for wounding the protective sheet.
8. The dispenser according to any of the preceding items, wherein the traction element comprises one or more toothed wheels.
9. The dispenser according to item 8, wherein said one or more toothed wheels is adapted to freewheel in one direction of rotation.
10. The dispenser according to any of the preceding items, wherein the switch comprises a slidable button and wherein activation of the switch includes sliding the slidable button.
11. The dispenser according to item 10, wherein activation of the switch corresponds to a single slide with the slidable button.
12. The dispenser according to item 10, wherein activation of the switch corresponds to two or more slides with the slidable button.
13. The dispenser according to item 10, wherein activation of the switch corresponds to sliding back and forth with the slidable button.
14. The dispenser according to any of items 10 to 13, wherein the switch is adapted to engage the traction element for only one sliding direction of the sliding button.
15. The dispenser according to any of the preceding items, wherein the traction mechanism further comprises a resilient switch release.
16. The dispenser according to item 15, wherein the resilient switch release is adapted to return the switch to a starting point after activation of the switch.
17. The dispenser according to any of the preceding items, wherein the traction mechanism is manually operated.
18. The dispenser according to any of the preceding items, wherein the traction mechanism is electronic.
19. The dispenser according to any of the preceding items, wherein the traction mechanism comprises a motor or actuator for driving the traction element.
20. The dispenser according to any of the preceding items, wherein said stack of adhesive elements is located adjacent said holder.
21. The dispenser according to any of the preceding items, wherein said stack of adhesive element is arranged vertically, such as located vertically below said holder.
22. The dispenser according to any of the preceding items, wherein said stack of adhesive element is arranged horizontally, such as located horizontally adjacent said holder.
23. The dispenser according to any of the preceding items, wherein the stack of adhesive elements is formed of adhesive elements sandwiched between layers of the protective sheet.
24. The dispenser according to any of the preceding items, wherein the adhesive elements are arranged exactly on top of each other in said stack.
25. The dispenser according to any of the preceding items, wherein the adhesive elements are displaced in relation to each other in said stack.
26. The dispenser according to any of the preceding items, wherein the protective sheet forms a band.
27. The dispenser according to item 26, each of said adhesive elements is arranged between two layers of protective sheet.
28. The dispenser according to item 26, each of said adhesive elements is arranged between two layers of protective sheet, said two layers formed by folding a single layer of protective sheet.
29. The dispenser according to any of the preceding items 26 to 28, wherein the stack of adhesive elements is formed such that the band of protective sheet can be released from the topmost or outermost adhesive element by pulling the band from only one side of the stack.
30. The dispenser according to any of the preceding items, wherein the resilient element comprises one or more springs.
31. The dispenser according to any of the preceding items, wherein the dispenser is adapted to automatically or at least semi-automatically arrange the adhesive element at a predetermined position at the medical device.
32. The dispenser according to any of the preceding items, wherein the medical device is an electrode assembly adapted to be arranged on the skin of a user.
33. The dispenser according to any of the preceding items, wherein the adhesive element is arranged on an electrode assembly of the medical device.
34. The dispenser according to any of the preceding items, wherein the dispenser is adapted to automatically or at least semi-automatically arrange the adhesive element at a predetermined position at the medical device.
35. The dispenser according to any of the preceding items, wherein the medical device is an electrode assembly adapted to be arranged on the skin of a user.
36. The dispenser according to any of the preceding items, wherein the adhesive element is arranged on an electrode assembly of the medical device.
37. The dispenser according to any of the preceding items, wherein the medical device is a part of the dispenser.
38. An adhesive element adapted to be arranged on a medical device.
39. The adhesive element according to any of the preceding items 38, wherein the adhesive element is double adhesive.
40. The adhesive element according to any of the preceding items 38 to 39, further adapted to match an attachment mechanism on the medical device.
41. The adhesive element according to any of the preceding items 38 to 40, wherein the adhesive element is adhesive on one side and comprises a part of an attachment mechanism on the opposite side, said part of said attachment mechanism adapted to match a corresponding part of an attachment mechanism on the medical device.
42. The adhesive element according to item 41, wherein the attachment mechanism is one, two, three or more snap locks.
43. The adhesive element according to any of items 41 to 42, wherein said part of said attachment mechanism comprises one or more components that are shaped to match a corresponding component of the attachment mechanism on the medical device.
44. The adhesive element according to item 43, wherein the shape of said components is polygonal, such as triangular, rectangular, pentagonal, hexagonal, or star-shaped.
45. The adhesive element according to any of items 41 to 44, wherein the attachment mechanism comprises hook and loop fasteners.
46. The adhesive element according to any of items 41 to 45, wherein the attachment mechanism comprises one or more clips or clamps.
47. The adhesive element according to any of items 41 to 46, wherein the attachment mechanism is at least partly magnetic.
48. The adhesive element according to any of items 41 to 47, wherein said attachment mechanism is adapted to provide an electrical connection between the adhesive element and the medical device.
49. The adhesive element according to any of the preceding items 38 to 48, wherein the adhesive elements are disposable.
50. The adhesive element according to any of the preceding items 38 to 49, wherein the adhesive elements are pads, such as gel pads.
51. The adhesive element according to any of the preceding items 38 to 50, wherein the shape of the adhesive element matches the shape of the medical device.
52. The adhesive element according to any of the preceding items 38 to 51, wherein the adhesive element is adapted to form an adhesive bond between the medical device and the skin of a user.
53. The adhesive element according to any of the preceding items 38 to 52, wherein the adhesive elements comprise conductive material.
54. The adhesive element according to any of the preceding items 38 to 53, wherein the adhesive element is flexible and/or bendable.
55. The adhesive element according to any of the preceding items 41 to 54, wherein the attachment mechanism is adapted migration between corresponding parts of the attachment mechanism.
56. The adhesive element according to any of the preceding items 41 to 55, wherein the attachment mechanism is adapted such that migration maintains electrical connection provided by the attachment mechanism.
57. The adhesive element according to any of the preceding items 41 to 56, wherein the attachment mechanism is adapted such that migration is provided in a direction substantially perpendicular to the skin when the medical device with adhesive element is applied to an individual.
58. The adhesive element according to any of the preceding items 38 to 57, wherein the adhesive element is adapted to form a conductive connection between the medical device and the skin of a user.
59. The dispenser according to any of the preceding items, wherein said adhesive element(s) is the adhesive element(s) according to any of items 38 to 58.
60. A tray for the dispenser according to any of the preceding items comprising a stack of adhesive elements arranged on a resilient element, said adhesive elements arranged in between layers of protective sheet, and a traction element connected to the protective sheet and adapted to pull said protective sheet.
61. The tray according to item 60, wherein said tray is replaceable or disposable.
62. The tray according to any of items 60 to 61, wherein said tray comprises any of the features described in any of the preceding items.
63. The tray according to any of items 60 to 62, wherein said adhesive element is the adhesive element defined by any of items 38 to 58.
64. A medical device for monitoring muscle activity of an individual comprising an electrode assembly with a plurality of electrodes, the electrode assembly comprising a part of an attachment mechanism adapted to match a corresponding part of an attachment mechanism on an adhesive element, wherein the medical device is adapted for receiving and monitoring electrical signals and providing electrical stimulation to said individual.
65. The medical device according to item 64, wherein the medical device is suitable for monitoring muscle activity related to bruxism, and adapted for processing the received signals in order to detect said bruxism, and wherein the electrical stimulation is a feedback signal provided in response to detection of the bruxism.
66. The medical device according to any of preceding items 64 to 65, wherein the attachment mechanism on the medical device comprises one or more clips or clamps.
67. The medical device according to any of preceding items 64 to 66, wherein said adhesive element is the adhesive element defined by any of items 38 to 58.
68. A kit comprising the medical device according to any of items 64 to 66 and the adhesive element defined by any of items 38 to 58, which are adapted to be attached to each other by means of the attachment mechanism.
69. A method for arranging a disposable adhesive element on a medical device comprising the steps of:
   a. activating the switch of the dispenser according to any of the preceding items thereby releasing the protective sheet from one side of the topmost adhesive element in the stack,
   b. arranging the medical device in the holder of said dispenser, the topmost adhesive element thereby being attached to the medical device, and
   c. removing the medical device with the adhesive element from the dispenser.
70. A method for arranging a disposable adhesive element on a medical device comprising the steps of:
   a. arranging the medical device in the holder of the dispenser according to any of the preceding items,
   b. activating the switch thereby releasing the protective sheet from the side of the topmost adhesive element in the stack that faces the medical device, the topmost adhesive element thereby being attached to the medical device, and
   c. removing the medical device with the adhesive element from the dispenser.

1-40. (canceled)

41. An adhesive element adapted to be arranged on a medical device wherein the adhesive element is a disposable gel pad adapted to match an attachment mechanism on the medical device that is adapted to provide an electrical connection between the adhesive element and the medical device and wherein the adhesive element comprises conductive material and is adapted to form a conductive connection between the medical device and the skin of a user.

42. The adhesive element according to claim 41, wherein the adhesive element is double adhesive.

43. The adhesive element according to claim 41, wherein the adhesive element is adhesive on one side and comprises a part of an attachment mechanism on the opposite side, said part of said attachment mechanism adapted to match a corresponding part of the attachment mechanism on the medical device.

44. The adhesive element according to claim 41, wherein the attachment mechanism is one, two, three or more snap locks.

45. The adhesive element according to claim 41, wherein said part of said attachment mechanism comprises one or more components that are shaped to match a corresponding component of the attachment mechanism on the medical device.

46. The adhesive element according to claim 41, wherein the shape of the adhesive element matches the shape of the medical device.

47. The adhesive element according to claim 41, wherein the adhesive element is adapted to form an adhesive bond between the medical device and the skin of a user.

48. The adhesive element according to claim 41, wherein the adhesive element is flexible and/or bendable.

49. The adhesive element according to claim 41, wherein the attachment mechanism is configured to allow migration between corresponding parts of the attachment mechanism.

50. The adhesive element according to claim 41, wherein the attachment mechanism is adapted such that migration maintains electrical connection provided by the attachment mechanism.

51. The adhesive element according to claim 41, wherein the attachment mechanism is adapted such that migration is provided in a direction substantially perpendicular to the skin when the medical device with adhesive element is applied to an individual.

52. A dispenser for arranging an adhesive element on a medical device, said dispenser comprising:
a holder adapted to receive the medical device;
a delivery mechanism comprising a stack of said adhesive elements and a resilient element, the stack arranged on the resilient element such that the stack of adhesive elements is pressed towards the holder, each of said adhesive elements arranged between layers of removable protective sheet; and
a traction mechanism comprising a traction element connected to the removable protective sheet and a switch adapted for operating the traction element, said traction mechanism adapted for pulling said removable protective sheet upon activation of the switch.

53. The dispenser according to claim 52, wherein the fraction mechanism is adapted for releasing the protective sheet from at least one side of an adhesive element, preferably the topmost adhesive element in the stack and preferably upon activation of the switch.

54. The dispenser according to claim 52, wherein the switch is adapted to mechanically engage the traction element, such as by means of one or more racks.

55. The dispenser according to claim 52, wherein the traction element comprises a cylindrical reel for wounding the protective sheet.

56. The dispenser according to claim 52, wherein the traction element comprises one or more toothed wheels configured to freewheel in one direction of rotation.

57. The dispenser according to claim 52, wherein the traction mechanism further comprises a resilient switch release configured to return the switch to a starting point upon activation of the switch.

58. The dispenser according to claim 52, wherein the stack of adhesive elements is formed of adhesive elements sandwiched between layers of the protective sheet and wherein the protective sheet forms a band.

59. The dispenser according to claim 58, wherein each of said adhesive elements is arranged between two layers of protective sheet, said two layers formed by folding a single layer of protective sheet.

60. The dispenser according to claim 58, wherein the stack of adhesive elements is formed such that the band of protective sheet can be released from the topmost or outermost adhesive element by pulling the band from only one side of the stack.

61. The dispenser according to claim 52, wherein said adhesive element(s) is an adhesive element adapted to be arranged on a medical device wherein the adhesive element is a disposable gel pad adapted to match an attachment mechanism on the medical device that is adapted to provide an electrical connection between the adhesive element and the medical device and wherein the adhesive element comprises conductive material and is adapted to form a conductive connection between the medical device and the skin of a user.

62. A medical device for monitoring muscle activity of an individual comprising an electrode assembly with a plurality of electrodes, the electrode assembly comprising a part of an attachment mechanism adapted to match a corresponding part of an attachment mechanism on the adhesive element defined by claim 41, wherein the medical device is adapted for receiving and monitoring electrical signals and providing electrical stimulation to said individual.

63. The medical device according to claim 62, wherein the medical device is suitable for monitoring muscle activity related to bruxism, and adapted for processing the received signals in order to detect said bruxism, and wherein the electrical stimulation is a feedback signal provided in response to detection of the bruxism.
